(19) United States
(12) Patent Application Publication
(10) Pub. No.: US 2020/0239473 A1
(43) Pub. Date: Jul. 30, 2020

(54) NEW DERIVATIVES OF LICOFELONE

(71) Applicant: Wolfgang ALBRECHT, Ulm (DE)

(72) Inventor: Wolfgang ALBRECHT, Ulm (DE)

(21) Appl. No.: 15/769,032

(22) PCT Filed: Oct. 20, 2016
(86) PCT No.: PCT/EP2016/075248

§ 371 Date: Apr. 17, 2018

(30) Foreign Application Priority Data
Oct. 21, 2015 (EP) 15190879.5

(51) Int. Cl. C07D 487/04

(52) U.S. Cl. CPC C07D 487/04; A61K 9/0053

(57) ABSTRACT

The present invention relates to novel compounds, e.g. for use as a medicament. In particular, the present invention relates to novel derivatives, preferably prodrugs, of licofelone suitable as a medicament, preferably in the treatment and/or prevention of systemic diseases, autoimmune diseases or inflammatory diseases. Further, the invention relates to a pharmaceutical composition comprising the novel compounds.

The present invention relates to novel compounds, preferably for use as a medicament. In particular, the present invention relates to novel derivatives, preferably prodrugs, of licofelone suitable as a medicament, preferably in the treatment and/or prevention of systemic diseases, autoimmune diseases and/or inflammatory diseases. Further, the invention relates to a pharmaceutical composition comprising the novel compounds.

### BACKGROUND OF THE INVENTION

The IUPAC name of licofelone is [6-(4-chlorophenyl)-2,2-dimethyl-7-phenyl-2,3-dihydro-1H-pyrrolizin-5-yl]acetic acid.

Licofelone is represented by the following chemical structure according to Formula (A):

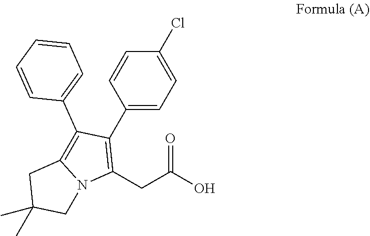 [SMILES: CC1(C)Cc2c(-c3ccccc3)c(-c3ccc(Cl)cc3)c(CC(=O)O)n2C1]

Licofelone is an analgesic and anti-inflammatory drug and differs from conventional nonsteroidal anti-inflammatory drugs (NSAIDs) and specific COX-2-inhibtitors in that it is an inhibitor of the cyclooxygenase (COX) enzymes COX-1 and COX-2 as well as an inhibitor of 5-lipogenase (5-LOX). The active pharmaceutical ingredient was developed for the symptomatic treatment of osteoarthritis.

Despite the different mechanisms of reaction, licofelone can be regarded as belonging to the class of NSAIDs, which, when used in higher doses, exhibit an anti-inflammatory effect. Michael Fine, “Quantifying the impact of NSAID-associated adverse events”, The American Journal of Managed Care, Vol. 19, no. Nov. 14, 2013, pages 267-272, reports that “nonsteroidal anti-inflammatory drugs (NSAIDs) are the cornerstone of pain management in patients who have inflammatory, acute pain (e.g. headache, postoperative pain, and orthopedic fractures) and chronic pain (e.g. rheumatoid arthritis, osteoarthritis, and gout).” Nevertheless, in order to reach an appropriate anti-inflammatory effect, the nonsteroidal anti-inflammatory drug like licofelone has to be administered in high doses. However, the use of high-dosed NSAIDs might cause undesired side-effects. For example, adverse effects such as an increased risk of kidney problems or in particular gastrointestinal (GI) problems are brought into interrelation with the administration of high-dosed NSAIDs. It is reported that licofelone shows improved gastro-intestinal tolerability compared to conventional NSAIDs (Bias P, Buchner A, Klesser B, Laufer S. 2004. Am J Gastroenterol. 99(4), 611-618). However upon chronic administration, dose-related adverse events are an issue.

Further, due to a strong food-effect and a high inter-individual variability licofelone is reported not to have an adequate bioavailability when administered orally. In addition, due to its low chemical stability the options for galenic formulations are limited. Up to now, most attempts to enhance the solubility/bioavailability result in a non-tolerable chemical degradation. Alternatively, known residues for the preparation of prodrugs of licofelone resulted in a poor or even no release of licofelone.

WO 2007/012464 A1 relates to a macrolide conjugated of pyrrolizine and indolizine (such as licofelone) with macrocyclic antibiotics. These pre-systemically stable conjugates were designed to preferentially deliver licofelone to immunocompetent white blood cells and not primarily to improve the pharmacokinetic properties. Furthermore, macrolides, including macrolide conjugates exhibit an interaction with cell membrane phospholipids which eventually damage the cell walls and cause phospholipidosis.

Thus, there is still a need for licofelone compounds with improved properties, preferably in the treatment and/or prevention of inflammatory diseases.

The licofelone compounds should be capable of being applied in doses high enough to show an appropriate anti-inflammatory effect. Further, the undesired side-effects associated with common NSAIDs should be reduced.

Further, the compounds should be sufficiently stable with regard to the formulation and the oral administration.

Additionally, the compounds should provide sufficient dissolution/bioavailability.

Hence, it was an object of the present invention to overcome the drawbacks of the above-mentioned available nonsteroidal anti-inflammatory drugs.

In particular it was an object to develop compounds to be used as a medicament for the above-mentioned diseases, wherein said compounds show an appropriate anti-inflammatory effect and wherein said compounds do not show undesired side-effects, such as gastrointestinal problems or kidney problems.

Further, licofelone should be provided in a form that shows sufficient stability and which provides enhanced dissolution/bioavailability.

### SUMMARY OF THE INVENTION

According to the present invention, the above objectives are achieved by the specific compounds described herein by Formula (I). Said compounds can be used as a medicament for the treatment and/or prevention of inflammatory diseases, for example osteoarthritis, rheumatoid arthritis, multiple sclerosis and psoriasis.

The subject of the present invention is a compound according to Formula (I)

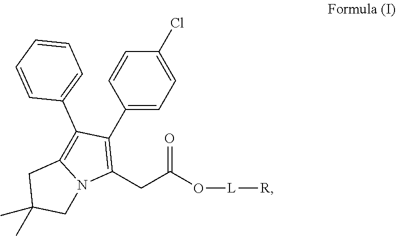

wherein
L is a linear organic residue comprising 2 to 30 carbon atoms, and
R is OH, —ONO₂ or —OOCR′, wherein —OOCR′ is a carboxylate group which is hydrolizable during small intestinal transit and/or wherein —OOCR′ can be the carboxylate of monocarboxylic acids or carboxylates of dicarboxylic acids and derivatives, such as monoesters, thereof, such as illustrated below;
or a pharmaceutically acceptable salt, hydrate, solvate, polymorph and/or mixtures thereof.

It was unexpectedly found that the compounds of the present invention show superior pharmaceutical and/or pharmacokinetic properties. Results from in vitro studies indicate that, in vivo, the compounds will be essentially quantitatively hydrolysed in the small intestine, which allows for a reliable absorption of licofelone. Additionally, the compounds exhibit an advantageous stability. Further the compounds can be advantageously formulated. Still further, the compounds show an advantageous ratio of desired anti-inflammatory effect to undesired side effects.

Further, the present invention relates to a compound according to Formula (I) or a pharmaceutically acceptable salt, hydrate, solvate, polymorph and/or mixtures thereof for use as a medicament, in particular for the treatment and/or prevention of inflammatory diseases, for example osteoarthritis, rheumatoid arthritis, multiple sclerosis and psoriasis.

Another subject is a pharmaceutical composition comprising the above-mentioned compound according to Formula (I).

### DETAILED DESCRIPTION OF THE INVENTION

In the context of this invention, the compound of the present invention is represented by above Formula (I). Further, the compound may refer to pharmaceutically acceptable salts, hydrates, solvates, polymorphs and mixtures thereof. For example, the invention also refers to pharmaceutically acceptable salts of compounds according to Formula (I) or to solvates of salts or hydrates. Further, the compound according to Formula (I) can be present in crystalline or amorphous form. If crystalline, all possible polymorphic forms are encompassed. The same applies to all embodiments, e.g. to compounds of Formulae (II) to (VI) as shown below.

In a preferred embodiment L can be a substituted or unsubstituted, linear, divalent aliphatic residue with 2 to 30 carbon atoms. More preferably L comprises 3 to 20 carbon atoms, or 4 to 15 or 5 to 12 or 6 to 10 carbon atoms or combinations thereof.

In case that L is substituted, the one or more substituents can preferably be selected independently from one or more of the following substituents: alkyl groups with 1 to 6 carbon atoms, halogen, nitro, nitrile, urea, phenyl, aldehyde, sulfate, amino, hydroxy, methoxy, mercapto, methylthio, phenyl and ═O, such that the corresponding —CO-group is formed.

Examples of an alkyl group with 1 to 6 are methyl, ethyl, propyl, isopropyl, butyl, tert.butyl, isobutyl, pentyl, neopentyl and hexyl.

Within the present application a divalent residue L can be regarded as a linking group being able to be bonded to two residues, wherein in the present case these residues are licofelone and R.

An aliphatic residue is a non-aromatic hydrocarbon compound which can comprise, apart from carbons and hydrogen atoms, for example also oxygen, sulphur and nitrogen atoms.

In the present invention L is a linear residue. The term “linear” shall exclude that L can be a cyclic residue. Further, in case that L is substituted, it is excluded that substituents are bonded with each other such that a cyclic residue is formed.

In a preferred embodiment L can be a substituted or unsubstituted alkylene group with 2 to 30 carbon atoms or a substituted or unsubstituted alkenylene group with 2 to 30 carbon atoms. Instead of the upper limit of 30 carbon atoms alternatively an upper limit of 6, 7, 8, 9, 10, 11, 12, 13, 14, 15, 16, 17, 18, 19, 20, 21, 22, 23, 24 or 25 can apply.

An alkylene group with 2 to 30 carbon atoms can be a linear alkylene group with 2 to 30 carbon atoms, preferably a linear alkylene group with 6 to 16 carbon atoms.

Alkylene groups with 2 to 30 carbon atoms can for example include ethylene, propylene, 2-methylpropylene, butylene, 2-methylbutylene, 3-methylbutylene, pentylene, sec.-pentylene, hexylene, ocytylene and dodecylene.

In a preferred embodiment L can be a substituted or unsubstituted alkylene group with 3 to 30 carbon atoms or a substituted or unsubstituted alkenylene group with 3 to 30 carbon atoms, wherein in the substituted or unsubstituted alkylene group with 3 to 30 carbon atoms and in the substituted or unsubstituted alkenylene group with 3 to 30 carbon atoms one or more —CH₂- group(s) can be substituted by an oxygen atom (—O-) to form an ether. Instead of the upper limit of 30 carbon atoms alternatively an upper limit of 6, 7, 8, 9, 10, 11, 12, 13, 14, 15, 16, 17, 18, 19, 20, 21, 22, 23, 24 or 25 can apply.

In a preferred embodiment L can be —((CH₂CHXO)ₘ-(CH₂CHX)- or —(CHXCH₂O)ₘ—(CHXCH₂)-, wherein m is 1 to 10, preferably 2 to 5, and wherein X is selected from methyl and ethyl. It is further preferred that m is 3 such that the corresponding L is —((CH₂CHXO)₃—(CH₂CHX)- or —(CHXCH₂O)₃—(CHXCH₂)-, wherein X is selected from methyl and ethyl.

In an alternatively preferred embodiment L can be —(CH₂)₃O)₃-(CH₂)₃-, wherein m is 0 to 10, preferably 1 to 9 and more preferably 2 to 8. In a more preferred embodiment L is —(CH₂)₂O)ₘ-(CH₂)₂-, and m is 2, 3, 4, 5 or 6, in particular m is 3.

In a preferred embodiment L can be —((CH₂)₂O)ₘ—(CH₂)₂-, wherein m is 0 to 10, preferably 1 to 9 and more preferably 2 to 8. In a more preferred embodiment L is —((CH₂)₂O)ₘ—(CH₂)₂-, and m is 2, 3, 4, 5 or 6, in particular m is 3, such that the corresponding L is —((CH₂)₂O)₃—(CH₂)₂-.

A preferred compound according to Formula (I) can thus be represented by the following Formula (II)

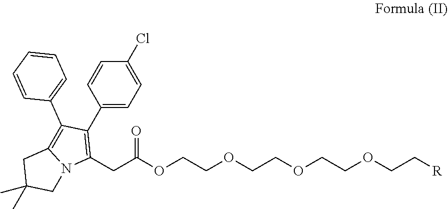

wherein R is OH, —ONO₂ or —OOCR′, wherein —OOCR′ is a carboxylate group which is hydrolizable during small intestinal transit
or a pharmaceutically acceptable salt, hydrate, solvate, polymorph and/or mixtures thereof.

In a further preferred embodiment R can be OH.

In a particularly preferred embodiment L can be —((CH₂)₂O)₃—(CH₂)₂- and R can be OH. Thus, a particularly preferred compound according to Formula (I) can be represented by the following Formula (III)

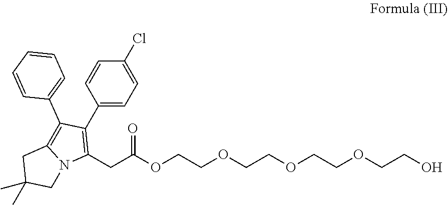 [SMILES: CC1(C)Cc2c(-c3ccccc3)c(-c3ccc(Cl)cc3)c(CC(=O)OCCOCCOCCOCCO)n2C1]

In a further preferred embodiment R can be —ONO₂. It is preferred that, in case R is —ONO₂, L is not a linear alkylene group.

In a particularly preferred embodiment L can be —((CH₂)₂O)ₘ-(CH₂)₂-, wherein m is 1 to 4 and R can be —ONO₂.

In an alternatively preferred embodiment R can be OOCR′, wherein OOCR′ is a carboxylate group which is hydrolizable during small intestinal transit.

In a particularly preferred embodiment L can be —((CH₂)₂O)₃—(CH₂)₂- and R can be OOCR′, wherein OOCR′ is a carboxylate group which is hydrolizable during small intestinal transit. Thus, a particularly preferred compound according to Formula (I) can be represented by the following Formula (IV)

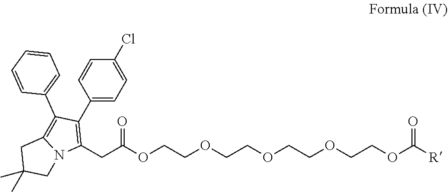

wherein OOCR′ is a carboxylate group which is hydrolizable during small intestinal transit.

The group R can be —OOCR′, wherein —OOCR′ is a carboxylate group which is hydrolizable during small intestinal transit.

A carboxylate group which is hydrolizable during small intestinal transit can be regarded a carboxylate group which is hydrolysed, preferably quantitatively hydrolysed during small intestinal transit. Hydrolysis is preferably conducted by the esterases and lipases in intestinal fluid and enterocytes. “During small intestinal transit” preferably refers to a contact with the intestinal fluid for 1 to 6 hours, preferably for 2 to 5 hours, in particular for 3 to 4 hours.

In a preferred embodiment the criteria “hydrolizable during small intestinal transit” is met, if the carboxylate group R′ of 1 gram compound is hydrolyzed in amounts of at least 80% when stirred at 250 rpm at 37° C. for 3 hours in 500 ml liquid containing:
100 ml minipig intestinal fluid
400 ml 2 wt.-% bovine serum albumin in FaSSIF.

The preparation of minipig intestinal fluid, the availability of bovine serum albumine and the composition of FaSSIF are described below in the experimental section. The amount hydrolysed is determined by LC-MS as described below in the experimental section.

In a preferred embodiment of the invention —OOCR′ can be the carboxylate of monocarboxylic acids, e.g. monocarboxylic acids having 2 to 6 carbon atoms, such as acetic acid, propanoic acid, lactic acid, butanoic acid, hexanoic acid or the carboxylate of dicarboxylic acids, e.g. having dicarboxylic acids having 3 to 10 carbon atoms, and derivatives, such as monoesters, thereof, such as succinic acid, fumaric acid, monomethyl fumarate, maleic acid, glutaric acid, adipic acid, pimelic acid and suberic acid.

In other words, a subject of the present invention is a compound according to Formula (I)

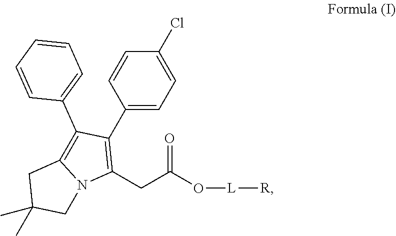

wherein
L is a linear organic residue comprising 2 to 30 carbon atoms, and
R is OH, —ONO₂ or —OOCR′, wherein -00CR′ is a carboxylate, provided that —OOCR′ is not the carboxylate of licofelone. Preferably, —OOCR′ is the carboxylate of monocarboxylic acids, e.g. monocarboxylic acids having 2 to 6 carbon atoms, such as acetic acid, propanoic acid, lactic acid, butanoic acid, hexanoic acid or the carboxylate of dicarboxylic acids, e.g. having dicarboxylic acids having 3 to 10 carbon atoms, and derivatives, such as monoesters, thereof, such as succinic acid, fumaric acid, monomethyl fumarate, maleic acid, glutaric acid, adipic acid, pimelic acid and suberic acid. In particular —OOCR′ is the carboxylate of monomethyl fumarate.

In a preferred embodiment of the present invention R can be the carboxylate of monomethyl fumarate (MMF). In case R is the carboxylate of MMF the above mentioned objects can especially advantageously be solved.

In a particularly preferred embodiment L can be —((CH₂)₂O)₃—(CH₂)₂- and R can be the carboxylate of monomethyl fumarate. A particularly preferred compound according to Formula (I) can thus be represented by the following Formula (V):

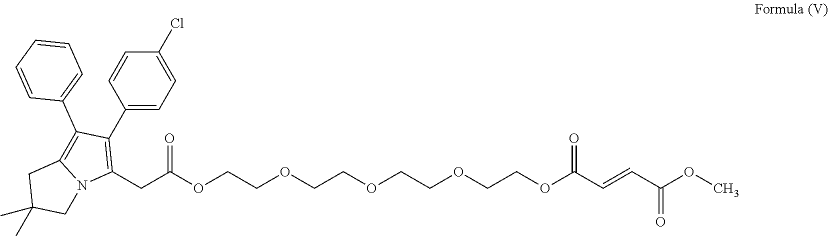

In an alternatively preferred embodiment R can be the carboxylate of an acid which in turn is coupled to further residue. For example in an alternatively particularly preferred embodiment L can be —((CH₂)₂O)₃—(CH₂)₂- and R can be the carboxylate of the acid represented by the following Formula (B):

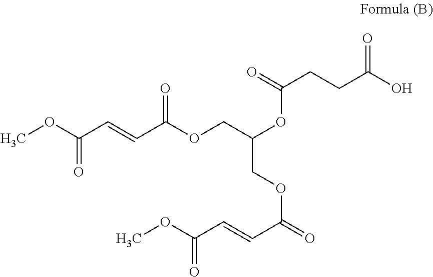 [SMILES: COC(=O)/C=C/C(=O)OCC(COC(=O)/C=C/C(=O)OC)OC(=O)CCC(=O)O]

The above acid can be regarded as succinic acid, wherein one carboxylic group is coupled via a linker to two MMF residues.

An alternatively particularly preferred compound according to Formula (I) can thus be represented by the following Formula (VI):

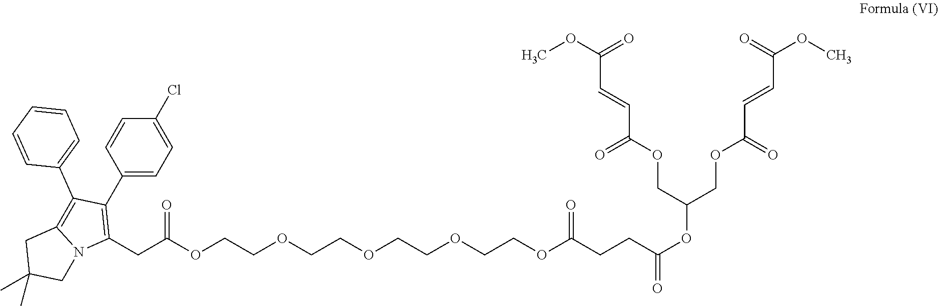

The above compounds according to Formula (I) show good anti-inflammatory properties. Additionally, the remaining organic residue once released in the gastro-intestinal lumen does not exhibit a pharmacological effect and will most likely quantitatively excreted without being absorbed.

A compound according to Formula (I) can preferably be synthesized via the following route:

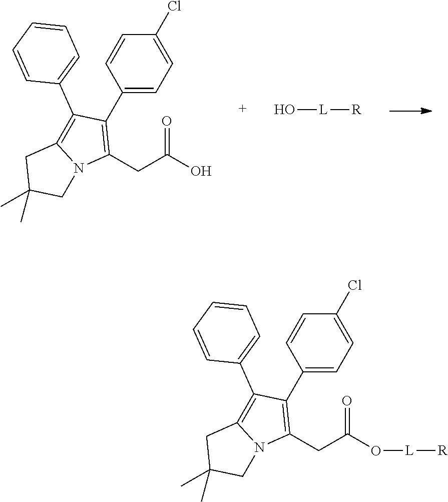

Preferably, HO-L-R and licofelone can be submitted to an esterification in an organic solvent in the presence of a coupling agent. A coupling agent is preferably a substance generally facilitating the formation of an ester or an amide. The coupling agent reacts with a carboxy group by forming a reactive intermediate which is subsequently further reacted with an alcohol or an amine to form the final product, i.e. an ester or an amide. Suitable coupling agents can be for example DCC (N,N′-dicyclohexylcarbodiimide), DIC (N,N′-diisopropylcarbodiimide), EDC×HCl (N-ethyl-N′-(3-methylaminopropyl)carbodiimide hydrochloride), CDI (carbonyldiimidazole), preferably EDC×HCl. It is further preferred that the coupling reaction is carried out in the presence of an auxiliary alkaline compound. Suitable alkaline compounds are for example pyridine and amines, such as triethylamine, diisopropylethylamine and DMAP (4-(dimethylamino)pyridine), in particular DMAP.

A suitable organic solvent can for example be dichloromethane, chloroform, acetonitrile, dioxane, tetrahydrofuran and dimethylformamide.

Alternatively, licofelone can be preferably reacted with thionyl chloride or oxalyl chloride, preferably oxalyl chloride, to form the corresponding acid chloride. Subsequently, the corresponding acid chloride can be submitted to a reaction with HO-L-R, preferably in an organic solvent, such as dioxane, tetrahydrofuran, chloroform or dichloromethane. Further, the reaction of the acid chloride with HO-L-R is preferably carried out in the presence of an auxiliary alkaline compound. Suitable alkaline compounds are for example pyridine and amines, such as triethylamine, DMAP (4-(dimethylamino)pyridine and diisopropylethylamine, preferably triethylamine.

Alternatively, the above acid chloride of licofelone can be further transferred in activated esters, like the para-nitrophenol ester.

Further alternatively, licofelone can be reacted with acid chlorides, diphenylphosphoryl azide or chlorosulfonyl isocyanate to form (mixed) anhydrides. These mixed anhydrides can be also submitted to further reactions to obtain further forms of anhydrides. For example, the anhydride of licofelone can be obtained by said preparation.

Subsequently, an activated ester or licofelone anhydride can be submitted to a reaction with HO-L-R, preferably in an organic solvent, such as dioxane, tetrahydrofuran, chloroform, acetone or dichloromethane. Further, the reaction of an activated ester or licofelone anhydride with HO-L-R is preferably carried out in the presence of an auxiliary alkaline compound. Suitable alkaline compounds are for example pyridine and amines, such as triethylamine, diisopropylethylamine and DMAP (4-(dimethylamino)pyridine), preferably DMAP.

Alternatively, the reaction of the activated ester or licofelone anhydride with HO-L-R can preferably be carried out in the absence of an auxiliary alkaline compound.

A suitable organic solvent can for example be dioxane, tetrahydrofuran and dimethylformamide. Alternatively, dichloromethane, acetonitrile and chloroform could be used. Further, mixtures thereof could be used.

In case that HO-L-R comprises more than one hydroxy group, one or more of these hydroxy groups can preferably be protected with a protection group before being submitted to a reaction with licofelone in the presence of a coupling agent, or with the acid chloride of licofelone, or with the anhydride of licofelone. Such a protection group can for example be a trialkylsilyl group.

After the coupling reaction, the protection can preferably be removed by a suitable reaction.

In an alternatively preferred embodiment a compound according to Formula (I) with R being —OOCR′ can preferably be synthesized via the following route:

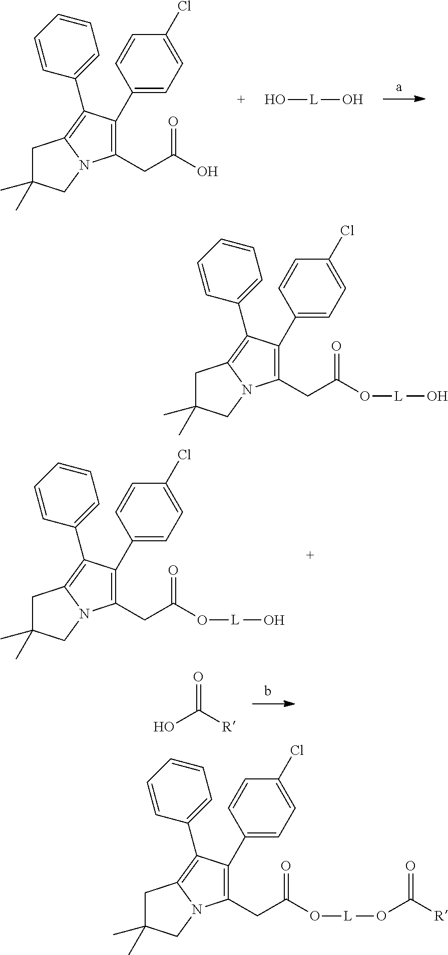

Preferably, in step a) HO-L-OH and licofelone can be submitted to an esterification in an organic solvent in the presence of a coupling agent. For step a) the same conditions apply as described above.

Preferably, in step b) the product from step a) and the HOOCR′ can be submitted to an esterification in an organic solvent in the presence of a coupling agent. For step b) the same conditions apply as described above.

Further, the present invention relates to a compound according to Formula (I) or a pharmaceutically acceptable salt, hydrate, solvate, polymorph and/or mixtures thereof for use as a medicament.

A further subject of the invention is a compound according to Formula (I) or a pharmaceutically acceptable salt, hydrate, solvate, polymorph and/or mixtures thereof for use in the treatment and/or prevention of systemic diseases, autoimmune diseases, inflammatory diseases, cancer and/or in the chondroprotection or chemoprevention.

To a compound according to Formula (I) for use in the treatment and/or prevention of systemic diseases, autoimmune diseases, inflammatory diseases, cancer and/or in the chondroprotection or chemoprevention the same applies as to a compound according to Formula (I) for use as a medicament.

Systemic diseases do not just affect single organs. Instead, these diseases are known to affect a number of organs and tissues or even the body as a whole.

People having an autoimmune disease usually suffer from their immune system mistakenly attacking the cells of their own organism and thus incorrectly responding to substances normally present in the body.

An inflammation can be defined as the response of the body to the occurrence of harmful stimuli which can result in pain, heat, redness, swelling and loss of function of the affected organ.

Cancer can be regarded as a group of diseases which are characterized by a non-normally rapid cell growth with the potential to invade or spread to other parts of the body. As a result the invaded part or organ might be damaged irreversibly and cease to fulfill its function.

Chondroprotection is the set of actions aimed at preventing, delaying or repairing degenerative joint lesions. Chondroprotection can be achieved by drugs that act to protect the joint cartilage, subchondral bone and synovial membrane such that the preserving of the integrity of cartilage, subchondral bone and synovial membrane might be achieved.

Chemoprevention can be regarded as a therapeutic technique in which bone marrow cells are removed from an individual with cancer and are genetically modified to withstand higher doses of chemotherapy before being returned to the donor.

It is possible that some of the above-mentioned diseases cannot be allocated in one single group of the above-mentioned groups since they show symptoms of more than one of them.

In a preferred embodiment the compounds of the present invention can be used in the treatment and/or prevention of osteoarthritis, rheumatoid arthritis, gout and psoriatic arthritis.

In a further aspect of the present invention it has been unexpectedly found that the above-mentioned compound according to Formula (I) can be advantageously used in the treatment and/or prevention of multiple sclerosis, rheumatoid arthritis and psoriasis, preferably multiple sclerosis. Said compounds can e.g. be used in the treatment of the following types of multiple sclerosis: relapsing-remitting, primary-progressive, secondary-progressive and progressive-relapsing. In a preferred embodiment the compounds of the present invention are used in the treatment of relapsing-remitting multiple sclerosis.

In said further aspect of the invention in compound according Formula (I), R can be —OOCR′, wherein —OOCR′ is a monomethyl fumarate residue. Such a compound can be regarded as a substance which is hydrolyzed—for example by esterases in the intestine—to monomethyl fumarate (MMF). Such a compound can be regarded as prodrug of MMF. MMF is reported also to be a metabolite of dimethyl fumarate DMF and can be characterized by the following chemical Formula (C):

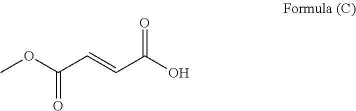

It is indicated that in vivo DMF and MMF show about the same efficacy, in particular on the transcription factor Nrf2. However, compared to DMF, the compound according Formula (I), wherein R is —OOCR′ and wherein —OOCR′ is an MMF residue shows advantageous pharmacokinetic properties. In particular, the present compounds are hydrolysed differently to MMF, for example more rapidly or more slowly, especially more slowly than DMF in the human body (or under respective in-vitro conditions) and cause fewer undesirable side effects than those associated with DMF.

Further, the present invention also provides a pharmaceutical composition comprising the compound according to the present invention, i.e. a pharmaceutical composition comprising a compound according to Formula (I) for use as a medicament and optionally pharmaceutical excipients.

In a preferred embodiment the pharmaceutical composition comprises
(i) 0.25 to 4 mmol, more preferably 0.3 to 2.5 mmol, still more preferably 0. 5 to 1.5 mmol and especially 1.05 mmol of a compound according to Formula (I) for use as a medicament
(ii) pharmaceutical excipient(s).

The parmaceutical formulation can preferably be further processed to or be in the form suitable for oral adminstration, preferably in form of a solid oral dosage form. Preferably the pharmaceutical composition is in form of a tablet or a capsule, in particular a tablet.

The pharmaceutical composition and/or the oral dosage form of the present invention can be prepared by methods well known to a person skilled in the art such as dry or wet granulation or direct compression.

The pharmaceutical composition can additionally contain one or more pharmaceutically acceptable excipient(s), such as fillers, binders, glidants, disintegrants and lubricants. Suitable excipients are for example disclosed in “Lexikon der Hilfsstoffe für Pharmazie, Kosmetik and angrenzende Gebiete”, published by H. P. Fielder, 4th Edition and “Handbook of Pharmaceutical Excipients”, 3rd Edition, published by A. H. Kibbe, American Pharmaceutical Association, Washington, USA, and Pharmaceutical Press, London.

The term filler generally means substances which serve to form the body of the tablet in the case of tablets with small amounts of active agent (e.g. less than 60% by weight). This means that fillers “dilute” the active agent(s) in order to produce an adequate tablet compression mixture. The normal purpose of fillers therefore is to obtain a suitable tablet size. Fillers may fulfil several requirements such as being chemically inert, non-hygroscopic, biocompatible, easily processable and possessing good biopharmaceutical properties. Fillers can be present in an amount of 0 to 80% by weight, preferably in an amount of 10 to 60% by weight based on the total weight of the composition.

A binder is generally a substance which is capable of increasing the strength of the resulting dosage form, especially the resulting tablets. Binders can be present in an amount of 0 to 30% by weight, preferably in an amount of 2 to 15% by weight based on the total weight of the composition.

Glidants can be used to improve the flowability. The glidant can be present for example in an amount of 0 to 3% by weight, preferably in an amount of 0.2 to 2% by weight based on the total weight of the composition.

Disintegrants are compounds which enhance the ability of the dosage form, preferably the ability of the tablet, to break into smaller fragments when in contact with a liquid, preferably water. The disintegrant can be present in an amount of 0 to 20% by weight, preferably in an amount of 1 to 15% by weight based on the total weight of the composition.

Lubricants are generally used in order to reduce sliding friction. In particular, the intention is to reduce the sliding friction found during tablet pressing between the punch moving up and down in the die and the die wall, on the one hand, and between the edge of the tablet and the die wall, on the other hand. Preferably, lubricants can be present in an amount of 0 to up to 5 wt %, more preferably of 0.1 to 4 wt % based on the total weight of the dosage form.

It is further preferred that the pharmaceutical composition is processed into an oral dosage form. The oral dosage form, preferably a tablet or a capsule, more preferably a tablet, can preferably be coated, preferably film coated.

In the present invention, the following three types of film coatings are possible:
film coating without affecting the release of the active ingredient,
gastric juice-resistant film coatings,
retard film coatings.

In a preferred embodiment a film coating without affecting the release of the active agent is used.

It is alternatively preferred that the present tablet is coated with a gastric juice-resistant film coating. Alternatively, a capsule comprising a gastric juice-resistant film coating can be used.

The gastric juice-resistant film coating preferably is a film coating being stable in the pH range of about 0.7 to 3.0, but in an environment with a pH value of 5 to 9 the gastric juice-resistant film coating preferably dissolves and the drug can be released.

The coating is preferably free of active ingredient. It is further preferred that the thickness of the coating is 1 μm to 2 mm, preferably 5 to 100 μm.

In a preferred embodiment the pharmaceutical composition can be administered one to three times a day, preferably once or twice a day, more preferably once a day.

Further, the present invention relates to a method for treating and/or preventing systemic diseases, autoimmune diseases and/or inflammatory diseases, preferably osteoarthritis and/or multiple sclerosis, rheumatoid arthritis or psoriasis, comprising administering to a subject in need thereof a therapeutically effective amount of the compound of the invention, in particular a compound according to Formula (I), wherein the residues are defined as above, or the pharmaceutical composition of the invention. Regarding the compounds and pharmaceutical compositions of the present invention the same explanations (e.g. regarding combination of possible embodiments) apply as described above. In an embodiment the treatment of multiple sclerosis is especially preferred.

The subjects and preferred embodiments of the present invention can be further illustrated by the following items:

1 Compound according to Formula (I)

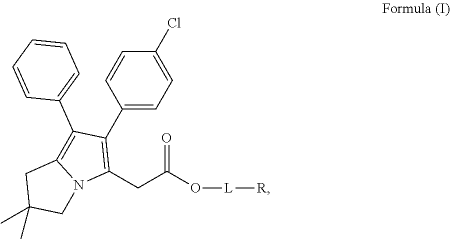

wherein
L is a linear organic residue comprising 2 to 30 carbon atoms, and

### R is —ONO₂.

2. Compound according to item 1, wherein L can be a substituted or unsubstituted divalent aliphatic residue with 2 to 30 carbon atoms.
3. Compound according to item 1 or 2, wherein L can be a substituted or unsubstituted alkylene group with 3 to 30 carbon atoms or a substituted or unsubstituted alkenylene group with 3 to 30 carbon atoms, wherein in the substituted or unsubstituted alkylene group with 3 to 30 carbon atoms or in the substituted or unsubstituted alkylene group with 3 to 30 carbon atoms one or more —CH₂- group(s) can be substituted by an oxygen atom (—O-) to form an ether.
4. Compound according to any one of items 1 to 3, wherein L is —((CH₂)₂O)ₘ—(CH₂)₂- wherein m is 1 to 10.
5. Compound according to item 4, wherein m is 3.
6. Compound according to any one of item 1 to 5 for use as a medicament.
7. Compound according to any one of the preceding items for use in the treatment of systemic diseases, autoimmune diseases or inflammatory diseases, preferably for the use in the treatment of osteoarthritis and/or multiple sclerosis, rheumatoid arthritis or psoriasis.
8. Pharmaceutical composition comprising a compound according to any one of the preceding items.
9. Pharmaceutical composition according to item 8, comprising
(i) 0.01 to 10 mmol of a compound according to any one of items 1 to 7 and
(ii) optionally pharmaceutical excipients.
10. Pharmaceutical composition according to item 8 or 9, wherein the composition is a solid oral dosage form.
11. Method for treating and/or preventing systemic diseases, autoimmune diseases and/or inflammatory diseases, preferably osteoarthritis and/or multiple sclerosis, rheumatoid arthritis or psoriasis, in particular multiple sclerosis, comprising administering to a subject in need thereof a therapeutically effective amount of the compound according to any one of claims 1 to 7 or the pharmaceutical composition according to any one of claims 8 to 10.

The invention is illustrated by the following examples.

### EXAMPLES

### Example 1

### (E)-But-2-enedioic acid 2-(2-hydroxy-ethoxy)-ethyl ester methyl ester

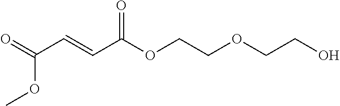

16.31 g (0.15 mol) diethylenglycol (DEG), 7.07 g (36.9 mmol) N-ethyl-N′-(3-dimethyl-aminopropyl)carbodiimide hydrochloride (EDC×HCl) and 0.19 g (1.5 mmol) 4-(dimethylamino)pyridine (DMAP) were dissolved in 40 ml tetrahydrofuran (THF). 4 g (30.8 mmol) monomethyl fumarate dissolved in 66 ml THF were dropped into the DEG solution at room temperature (RT) within 35 minutes. The reaction mixture was kept under continuous stirring at RT for 1.5 h. Stirring was stopped and a biphasic system was obtained; the lower layer was discarded and the upper layer was evaporated. The obtained crude product (colorless oil) was subjected to flash chromatography (100% ethyl acetate) twice. The product was dried under high vacuum at RT for 5 hours to yield the product as colorless oil (2.7 g; 12.3 mmol).

¹ NMR (400 MHz, CDCl₃) δ [ppm]: 2.44-2.49 (s, 1 H) 3.53-3.59 (m, 2 H) 3.69 (s, 4 H) 3.75 (s, 3 H) 4.31 (m, J=4.70, 4.70 Hz, 2 H) 6.82 (s, 2H)

### Example 2

### (E)-But-2-enedioic acid 2-{2-[2-(2-hydroxy-ethoxy)-ethoxy]-ethoxy}-ethyl ester methyl ester

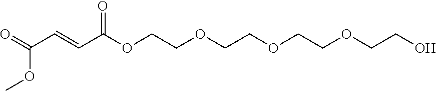 [SMILES: COC(=O)/C=C/C(=O)OCCOCCOCCOCCO]

13.4 g (69.2 mmol) Tetraethylenglycol (TEG), 5.3 g (27.7 mmol) EDC×HCl, 0.14 g (1.2 mmol) DMAP were dissolved in 50 ml THF. 3 g (23 mmol) monomethyl fumarate, dissolved in 50 ml THF, were added into the TEG solution at RT. The reaction mixture was kept under continuous stirring at RT for 3 h. Stirring was stopped and a biphasic layer was obtained, the lower layer was discarded and the upper layer evaporated. The obtained crude product was subjected to flash chromatography (100% ethyl acetate) twice. The product was dried under high vacuum at RT for 5 hours to yield the product as colorless oil (3.14 g; 10.3 mmol)

¹H NMR (400 MHz, CDCl₃) δ [ppm]: 2.53 (s, 1 H) 3.55-3.60 (m, 2 H) 3.64 (s, 8 H) 3.67-3.74 (m, 4 H) 3.78 (s, 3 H) 4.32-4.35 (m, 2H) 6.86 (s, 2 H)

### Example 3

### (E)-But-2-enedioic acid 2-{2-[2-(2-{2-[2-(4-chloro-phenyl)-6,6-dimethyl-1-phenyl-6,7-dihydro-5H-pyrrolizin-3-yl]-acetoxy}-ethoxy)- ethoxy]-ethoxy}-ethyl ester methyl ester

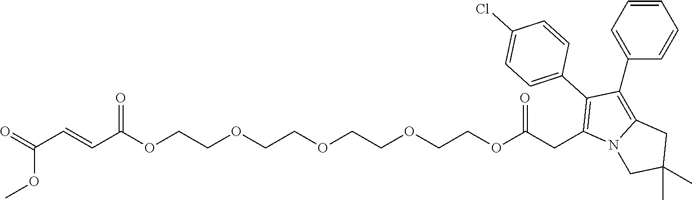 [SMILES: COC(=O)/C=C/C(=O)OCCOCCOCCOCCOC(=O)Cc1c(-c2ccc(Cl)cc2)c(-c2ccccc2)c2n1CC(C)(C)C2]

1 g (2.6 mmol) 6-(4-chlorophenyl)-2,2-dimethyl-7-phenyl-2,3-dihydro-1H-pyrrolizin-5-yl]acetic acid (licofelone), 0.6 g (3.2 mmol) EDC×HCl , 20 mg (0.1 mmol) DMAP and 0.89 g (2.9 mmol) (E)-But-2-enedioic acid 2-{2-[2-(2-hydroxy-ethoxy)-ethoxy]-ethoxy}-ethyl ester methyl ester were dissolved in 30 ml THF. During O/N stirring at RT, a bright yellow solution with a syrupy white precipitate was formed. The solvent was evaporated, to the bright yellow syrup 50 ml water were added and the aqueous layer was extracted with 3×100 ml ethyl acetate. The organic layers were combined, the solvent was evaporated and the crude product subjected to flash chromatography (ethyl acetate:n-heptane 50:50 (v/v)) to yield the product as yellow oil, which was dried at 17 mbar at room temperature for 5 hours to afford the product as yellow syrupy product (1.1 g; 1.6 mmol).

¹H NMR (400 MHz, CDCl₃) δ [ppm]: 1.29 (s, 6 H) 2.16 (s, 1 H) 2.84 (s, 2 H) 3.55 (s, 2 H) 3.63 (d, J=2.35 Hz, 8 H) 3.68-3.76 (m, 6 H) 3.80 (s, 3H) 4.25-4.30 (m, 2 H) 4.32-4.36 (m, 2 H) 6.88 (s, 2 H) 7.02-7.09 (m, 3 H) 7.11 - 7.15 (m, 2 H) 7.18 (s, 2 H) 7.21-7.27 (m, 2 H)

LC-MS: tᵣ: 12.3 min.; m/z: 688 [M+H]⁺ (method C)

### Example 4a

### [2-(4-Chloro-phenyl)-6,6-dimethyl-1-phenyl-6,7-dihydro-5H-pyrrolizin-3-yl]-acetic acid 2-{2-[2-(2-hydroxy-ethoxy)-ethoxy]-ethoxy}-ethyl ester (4a) and

### Reference Example 4b

### [2-(4-Chloro-phenyl)-6,6-dimethyl-1-phenyl-6,7-dihydro-5H-pyrrolizin-3-yl]acetic acid 2-{2-[2-(2-{2-[2-(4-chloro-phenyl)-6,6-dimethyl-1-phenyl-6,7-dihydro-5H-pyrrolizin-3-yl]-acetoxy}-ethoxy)-ethoxy]-ethoxy}-ethyl ester (4b)

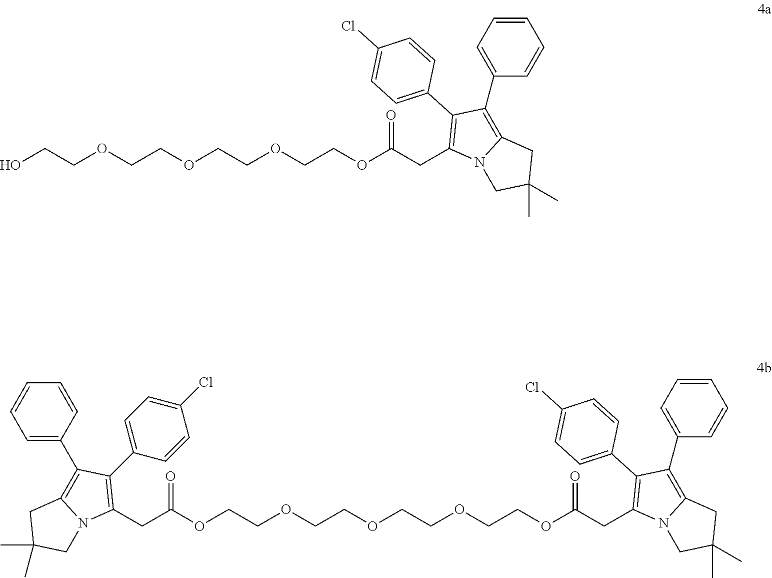

1.53 g (7.9 mmol) tetraethylenglykol, 1.21 g (6.3 mmol) EDC×HCl, 30 mg (0.3 mmol) DMAP and 2 g (5.3 mmol) 6-(4-chlorophenyl)-2,2-dimethyl-7-phenyl-2,3-dihydro-1H-pyrrolizin-5-yl]acetic acid (licofelone) were dissolved in 40 ml THF. The reaction mixture was stirred for 6 hours. Stirring was stopped (bright yellow solution with a syrupy white precipitate) and the organic layer was decanted off from the syrupy product. To the residue 40 ml THF were added and the mixture was stirred for 1 minute before being decanted off again. The solvent of the combined THF layers was evaporated, yielding a yellow syrup. The crude product contained two major products, 4a and 4b. After flash chromatography (gradient: ethyl acetate/n-heptane 1:2 (v/v) -> ethyl acetate/n-heptane 2:1 -> ethyl acetate (100%) to yield the above products:

4b (0.76 g (0.8 mmol)) was obtained as a colorless solid (R_{f} (ethyl acetate/n-heptane 2:1): 0.63). The product was dried at 7×10⁻² mbar at RT for 2 hours.

¹H NMR (400 MHz, acetone-d₆) δ [ppm]: 1.29 (s, 12 H) 2.80-2.82 (m, 5 H) 3.55 (s, 4 H) 3.58 (s, 8 H) 3.66-3.70 (m, 4 H) 3.81 (s, 4 H) 4.20-4.26 (m, 4 H) 7.02-7.07 (m, 6 H) 7.14-7.19 (m, 8 H) 7.29 (d, J=8.21 Hz, 4 H)

LC-MS: tᵣ: 15.0 min.; m/z: 917 [M+H]⁺ (method D)

4a (R_{f} EtOAc/H 2/1: 0.12) was obtained as slightly brown to yellow oil. After a second chromatography (silica, 100% ethyl acetate), 4a was obtained as a yellow oil (1.06 g; 0.8 mmol). The product was dried at 7×10⁻² mbar at RT for 2 hours.

¹H NMR (400 MHz, acetone-d₆) δ [ppm]:1.30 (s, 6 H) 2.79-2.84 (m, 3 H) 3.44-3.56 (m, 3 H) 3.57 (s, 6 H) 3.59-3.61 (m, 5 H) 3.69-3.72 (m, 2 H) 3.83 (s, 2 H) 4.23-4.27 (m, 2H) 7.03-7.08 (m, 3H) 7.14-7.20 (m, 4H) 7.31 (d, J=8.60 Hz, 2H)

LC-MS: tᵣ:10.7 min.; m/z: 556 [M+H]⁺ (method D)

### Example 5a

### 2-hydroxyethyl 2-[2-(4-chlorophenyl)-6,6-dimethyl-1-phenyl-5,7-dihydropyrrolizin-3-yl]acetate (and

### Reference Example 5b

### 2-[2-[2-(4-chlorophenyl)-6,6-di-methyl-1-phenyl-5,7-dihydropyrrolizin-3-yl]acetyl]oxyethyl-2-[2-(4-chloro-phenyl)-6,6-dimethyl-1-phenyl- 5,7-dihydropyrrolizin-3-yl]acetate

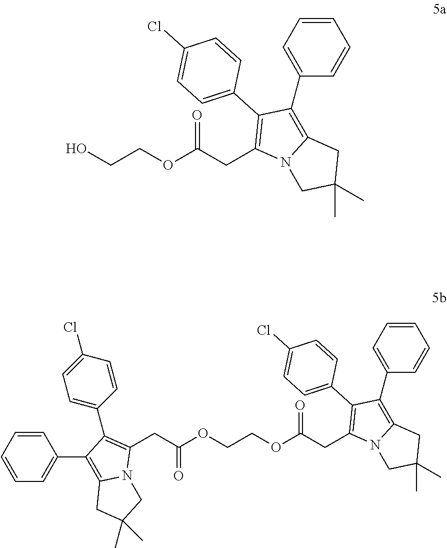 [SMILES: CC1(C)Cc2c(-c3ccccc3)c(-c3ccc(Cl)cc3)c(CC(=O)OCCO)n2C1 ; CC1(C)Cc2c(-c3ccccc3)c(-c3ccc(Cl)cc3)c(CC(=O)OCCOC(=O)Cc3c(-c4ccc(Cl)cc4)c(-c4ccccc4)c4n3CC(C)(C)C4)n2C1]

In a 100 ml RBF, 1.77 ml (31.6 mmol) ethylene glycol, 1,82 g (9,5 mmol) EDC×HCl and 50 mg (0.4 mmol) DMAP were dissolved in 20 ml THF. 3 g (7.9 mmol) (2.6 mmol) 6-(4-chlorophenyl)-2,2-dimethyl-7-phenyl-2,3 -dihydro-1H-pyrrolizin-5-yl]acetic acid (licofelone) dissolved in 30 ml THF were dropped into this solution within 55 min. The reaction mixture was kept under continuous stirring at RT for two days. Stirring was stopped and the solvent was evaporated. The crude product contained two major products, 5a and 5b. Separation was achieved by flash chromatography (ethyl acetate/n-heptane 50:50 (v/v)).

Fraction 1 contained compound 5b, which was obtained as a slightly yellow oil after solvent evaporation. After addition of 30 ml n-pentane and overnight stirring, a white precipitate was obtained, which was filtered off and dried for 1 hour at 50° C. and 23 mbar.

¹H-NMR (400 MHz, DMSO-d₆) δ ppm: 1.17 (s, 6 H) 2.75 (s, 2 H) 3.53 (s, 2 H) 3.67 (s, 2 H) 4.29 (s, 2 H) 6.94 (d, J=7.43 Hz, 2 H) 6.99-7.06 (m, 3 H) 7.14 (t, J=7.04 Hz, 2 H) 7.27 (d, J=8.21 Hz, 2 H)

LC-MS (ESI⁺): m/z 785 [M+H]⁺

Fraction 2 contained compound 5a, which solidified as a foam after solvent evaporation.

¹H-NMR (400 MHz, DMSO-d6) δ ppm: 1.21 (s, 6 H) 2.76 (br. s., 2 H) 3.53 (br. s., 2 H) 3.56 (d, J=4.69 Hz, 2 H) 3.72 (br. s., 2 H) 4.02-4.12 (m, 2 H) 4.75-4.85 (m, 1 H) 6.91-6.99 (m, 2H) 7.00-7.10 (m, 3 H) 7.12-7.20 (m, 2H) 7.27-7.35 (m, 2H)

LC-MS (ESI⁺): m/z 424 [M+H]⁺

### Example 6

### O4-[2-[2-[2-(4-chlorophenyl)-6,6-dimethyl-1-phenyl-5,7-dihydro-pyrrolizin-3-yl]acetyl]oxyethyl] O1-methyl (E)-but-2-enedioate

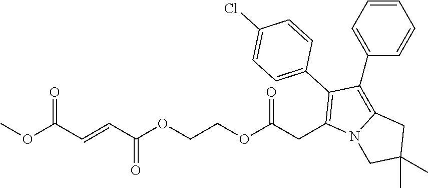

1.5 g (3.5 mmol) 2-hydroxyethyl 2-[2-(4-chlorophenyl)-6,6-dimethyl-1-phenyl-5,7-dihydropyrrolizin-3-yl]acetate, 1 g (5.2 mmol) EDC×HCl, 20 mg (0.2 mmol) DMAP and 0.55 g (4.2 mmol) monomethyl fumarate were dissolved in 20 ml THF. The reaction mixture was stirred 0/N at RT. Stirring was stopped (solution with a syrupy brown precipitate) and the solvent was evaporated yielding a brown syrup. After addition of 100 ml water the mixture was extracted with 3×100 ml ethyl acetate. The solvent was evaporated and the crude product subjected to flash chromatography (80 g silica gel, ethyl acetate/n-heptane 50:50 (v/v), flow 30 mL/min.) to yield the product as yellow oil, which was dried at 4×10⁻² mbar at RT for 1 hour. To the oily product, 30 ml n-pentane were added and the mixture was stirred O/N at RT which resulted in the formation of a precipitate. The solid was filtered off and dried under vacuum at 30° C. for 30 minutes.

¹H-NMR (400 MHz, DMSO-d₆) δ [ppm]: 1.20 (s, 6 H) 2.76 (s, 2 H) 3.55 (s, 2 H) 3.70 (s, 5 H) 4.30-4.39 (m, 4 H) 6.73 (s, 2 H) 6.94 (d, J=7.43 Hz, 2 H) 7.00-7.07 (m, 3 H) 7.12-7.18 (m, 2 H) 7.29 (d, J=7.82 Hz, 2 H).

LC-MS (ESI⁺): m/z 536 [M+H]⁺

### Example 7

### (E)-But-2-enedioic acid 2-(2-{2-[2-(4-chloro-phenyl)-6,6-dimethyl-1-phenyl-6,7-dihydro-5H-pyrrolizin-3-yl]-acetoxy}-ethoxy)-ethyl ester methyl ester

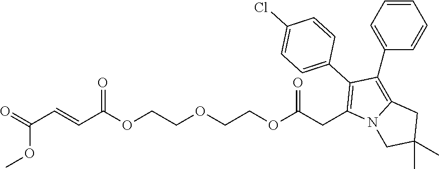 [SMILES: COC(=O)/C=C/C(=O)OCCOCCOC(=O)Cc1c(-c2ccc(Cl)cc2)c(-c2ccccc2)c2n1CC(C)(C)C2]

1 g (2.6 mmol) 6-(4-chlorophenyl)-2,2-dimethyl-7-phenyl-2,3-dihydro-1H-pyrrolizin-5-yl]acetic acid (licofelone), 0.6 g (3.2 mmol) EDC×HCl, 0.02 g (0.1 mmol) 4-(dimethylamino)pyridine (DMAP) and 0.632 g (2.9 mmol) (E)-But-2-enedioic acid 2-(2-hydroxy-ethoxy)-ethyl ester methyl ester (were dissolved in THF (10 ml). The reaction mixture was kept under continuous stirring at room temperature for ˜1.5 h. Stirring was stopped (bright yellow solution with a syrupy white precipitate) and the solvent was evaporated, yielding a bright yellow syrup. Water (50 ml) was added and the aqueous layer was extracted with ethyl acetate 3 times (3×100 ml). The solvent was evaporated and the crude product subjected to flash chromatography (dichloromethane/MeCN 4:1) to yield the product as yellow oil, which was dried at 7×10⁻² mbar at RT for 5 hours to afford the product as a yellow syrupy product (1.01 g; 1.7 mmol).

¹H NMR (400 MHz, acetone-d₆) δ [ppm]: 1.28-1.32 (m, 6 H) 2.79-2.83 (m, 2 H) 3.58 (s, 2H) 3.72-3.80 (m, 7 H) 3.82 (s, 2 H) 4.24-4.28 (m, 2H) 4.32-4.35 (m, 2 H) 6.79 (s, 2 H) 7.03-7.07 (m, 3 H) 7.14-7.19 (m, 4 H) 7.30 (d, J=8.21 Hz, 2 H)

LC-MS: tᵣ: 13.5 min.; m/z: 580 [M+H]⁺ (method B)

### Example 8

### rac-[2-(4-Chloro-phenyl)-6,6-dimethyl-1-phenyl-6,7-dihydro-5H-pyrrolizin-3-yl]-acetic acid 2,3-dihydroxy-propyl ester

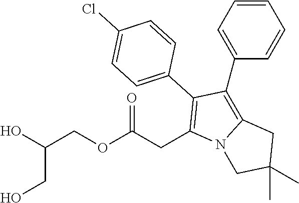 [SMILES: CC1(C)Cc2c(-c3ccccc3)c(-c3ccc(Cl)cc3)c(CC(=O)OCC(O)CO)n2C1]

Step 1: Synthesis of rac-[2-(4-chloro-phenyl)-6,6-dimethyl-1-phenyl-6,7-dihydro-5H-pyrrolizin-3-yl]-acetic acid 2,2-dimethyl-[1,3]dioxolan-4-ylmethyl ester

1.74 g (13.2 mmol) rac-(2,2-dimethyl-[1,3]dioxolan-4-yl)-methanol, 1.82 g (9.5 mmol) EDC×HCl and 0.05 g (0.4 mmol) DMAP were dissolved in 15 ml THF. 3 g (7.9 mmol) 6-(4-chlorophenyl)-2,2-dimethyl-7-phenyl-2,3 -dihydro-1H-pyrrolizin-5-yl]acetic acid (licofelone) dissolved in 20 ml THF were dropped into this solution within 15 min. The reaction mixture was kept under continuous stirring at RT for 3 hours. Stirring was stopped and the solvent was decanted off. The decanted organic layer was evaporated and the obtained yellow oily product was subjected to flash chromatography (ethyl acetate/n-heptane 1:2 (v/v)) to yield the oily product. To the yellow oil 50 ml n-pentane were added while stirring. A slightly yellow to white solid precipitated. After stirring for 1 hour, the product was filtered off and dried for 2 hours at RT at 21 mbar.

¹H NMR (400 MHz, acetone-d₆) δ [ppm]: 1.30 (s, 5 H) 1.34 (s, 1 H) 2.80-2.83 (m, 1 H) 3.60 (s, 1 H) 3.74 (dd, J=8.41, 6.06 Hz, 0.5 H) 3.82 (s, 1 H) 4.07 (dd, J=8.21, 6.65 Hz, 0.5 H) 4.17 (dd, J=5.08, 3.52 Hz, 1 H) 4.32 (d, J=4.69 Hz, 0.5 H) 7.03 - 7.07 (m, 1 H) 7.15-7.19 (m, 2 H) 7.30 (d, J=8.21 Hz, 1 H)

Step 2: Synthesis of rac-[2-(4-chloro-phenyl)-6,6-dimethyl-1-phenyl-6,7-dihydro-5H-pyrrolizin-3-yl]-acetic acid 2,3-dihydroxy-propyl ester

Under nitrogen atmosphere 7.5 ml trifluoro acetic acid, dissolved in 7.5 ml DCM were added to 1 g of the product from Step 1 and the mixture was stirred overnight at RT. 100 ml water were added. The aqueous layer was neutralized with saturated disodium carbonate solution, then the aqueous layer was extracted with 2×100 ml ethyl acetate, the combined organic layers were dried over sodium sulfate and evaporated. The crude mixture (brown oil) was subjected to flash chromatography (80 g silica, ethyl acetate/n-heptane 2:1 (v/v), flow 35 ml/min.) to yield 0.143 g (0.3 mol) of the target compound.

¹H NMR (400 MHz, DMSO-d₆) δ [ppm]: 1.22 (s, 6 H) 2.77 (s, 2 H) 3.32-3.40 (m, 3 H) 3.54 (s, 2 H) 3.64-3.70 (m, 1 H) 3.74 (s, 2 H) 3.99 (dd, J=10.95, 6.65 Hz, 1 H) 4.09-4.15 (m, 1 H) 4.63-4.69 (m, 1 H) 4.92 (d, J=5.08 Hz, 1 H) 6.97 (d, J=7.04 Hz, 2 H) 7.00-7.05 (m, 1 H) 7.08 (d, J=8.60 Hz, 2 H) 7.13-7.18 (m, 2 H) 7.31 (d, J=8.60 Hz, 2 H)

LC-MS: tᵣ:7.0 min.; m/z: 492 [M+K]⁺, 476 [M+Na]⁺, 454 [M+H]⁺ (method F)

### Analytical Methods:

### Nuclear Magnetic Resonance Spectroscopy:

Instrument: Varian Mercury 400 Plus NMR Spectrometer, Oxford AS, 400 MHz.

### HPLC/UV/(Ion Trap) MS:

Instrument: Agilent, HP 1200
Column: Phenomenex Kinetex C18, 150*4.6 mm, 2.6 μm
Oven temperature: 40° C.
Injection volume: 3 μl
Flow: 0.8 ml/min (method A, E, F); 1.0 ml/min (methods B, C and D)
   coupled with:
Instrument: Esquire HCT (Broker Daltonics)

### MS Parameters:

Dry temperature: 320° C.
Nebulizer: 65.0 psi
Dry gas: 8.0 l/min
Ionization (polarity): electrospray (positive)
Scan range: m/z 50-1000

### HPLC Methods:

### Method B

Solvent A: acetonitrile
Solvent B: 0.1% formic acid, HFBA pH=4

| time [min] | solvent B [%] |
|---|---|
| 0.00 | 80 |
| 2.00 | 80 |
| 10.00 | 30 |
| 11.00 | 15 |
| 15.00 | 15 |
| 15.10 | 80 |
| 22.00 | 80 |

### Method C

Solvent A: acetonitrile; 0.2% formic acid; 0.1% HFBA
Solvent B: 0.2% formic acid, 0.1% HFBA

| time [min] | solvent B [%] |
|---|---|
| 0.00 | 60 |
| 15.00 | 5 |
| 16.00 | 5 |
| 16.10 | 60 |
| 22.00 | 60 |

### Method D

Solvent A: acetonitrile; 0.2% formic acid; 0.1% HFBA
Solvent B: 0.2% formic acid, 0.1% HFBA

| time [min] | solvent B [%] |
|---|---|
| 0.00 | 45 |
| 15.00 | 5 |
| 16.00 | 5 |
| 16.10 | 45 |
| 22.00 | 45 |

### Method F

Solvent A: acetonitrile
Solvent B: 0.2% formic acid, 0.1% HFBA

| time [min] | solvent B [%] |
|---|---|
| 0.00 | 40 |
| 3.00 | 40 |
| 7.00 | 15 |
| 10.50 | 15 |
| 10.60 | 40 |
| 17.00 | 40 |

### Example 9

### Metabolic Stability of NSAID in Minipig Intestinal Fluid—Quantification of Licofelone

### a) Preparation and Storage of Minipig Intestinal Fluid (IF)

Intestinal tissue/fluid and enterocyte samples were taken from a 10 month old, female Gottingen SPF minipig. The body weight was 21 kg. The minipig was fasted for approximately 28 hours before sampling of intestinal fluid/tissues and enterocytes. On the day of sampling, the minipig was weighed and anaesthetised. The animal was killed by exsanguination before the sampling of intestinal fluid. An intestinal segment was ligated at both ends before removal. The isolated tissue was placed in isotonic saline and opened by a longitudinal cut for sampling of intestinal fluid.

The intestinal tissue from each segment was transferred into a Centrifuge Tube, immersed in 10 ml 50mM phosphate buffer, pH 6.8 and frozen at −70° C.

b) Incubation Experiments with Minipig Intestinal Fluid (without FaSSIF)

In a HPLC glass vial, 8 μl of stock solution were mixed with 792 μl diluted IF (1 vol IF+4 vol 2% bovine serum albumin in FaSSIF) and the mixture was stirred (250 rpm) in a water bath (T=37° C.).

Immediately after mixing as well as at t=30 min, 60 min, 120 min and 240 min, 50 μl were withdrawn, diluted with 100 μl acetonitrile, vortexed for 15 sec and centrifuged (13000 rpm, 3 min). 5 μl of supernatant were injected for HPLC/UV analysis.
column: Acquity UPLC BEH C18, 50×2.1 mm i.d., dp=1.7
oven temperature: 40° C.
flow: 0.5 ml/min
solvent A: Acetonitrile
solvent B: 20 mM KH₂PO₄, pH 4.25

| gradient: | time [min] | solvent B [%] |
|---|---|---|
| | 0.00 | 40 |
| | 2.00 | 40 |
| | 6.00 | 15 |
| | 10.00 | 15 |
| | 10.10 | 40 |
| | 15.00 (stoptime) | 40 |

detection: DAD (λ=248 nm (60 mm cell))

Fasted State Simulated Intestinal Fluid (FaSSIF) contains:
Sodium taurocholate 3 mM
Lecithin 0.75 mM
NaOH 0.174 g
NaH2PO4.H2O 1.977 g
NaCl 3.093 g
Purified water qs. 500 mL
Media has a pH of 6.50 and an osmolality of about 270 mOsmol/kg.

Preparation is described in Dissolution Technologies, May 2004, Page 16.

Bovine serum albumin can be purchased from Sigma Aldrich, Product A3675, Bovine Serum Albumin lyophilized powder, low endotoxin, >98% (agarose gel electrophoresis).

### c) Quantification of Licofelone by HPLC/UV

A set of six reference solutions (solvent: 1 vol IF and 4 vol 2% bovine serum albumin in FaSSIF pH 6.5) with licofelone concentrations between 38.0 and 0.19 μg/ml and the solvent without licofelone were analysed in duplicate. The obtained concentration/peak area data pairs were subjected to linear regression analysis and the resulting calibration curve (r²=0.9999) was used for quantification of licofelone in incubation experiments.

### d) Results

Incubation of the licofelone compounds as produced in Examples 2 to 11 in diluted minipig intestinal fluid resulted in an unexpected smooth cleavage of the ester bond and thus in a release of the individual moieties, such as licofelone and monomethyl fumarate. The analytical method allowed for quantification of the release of licofelone.

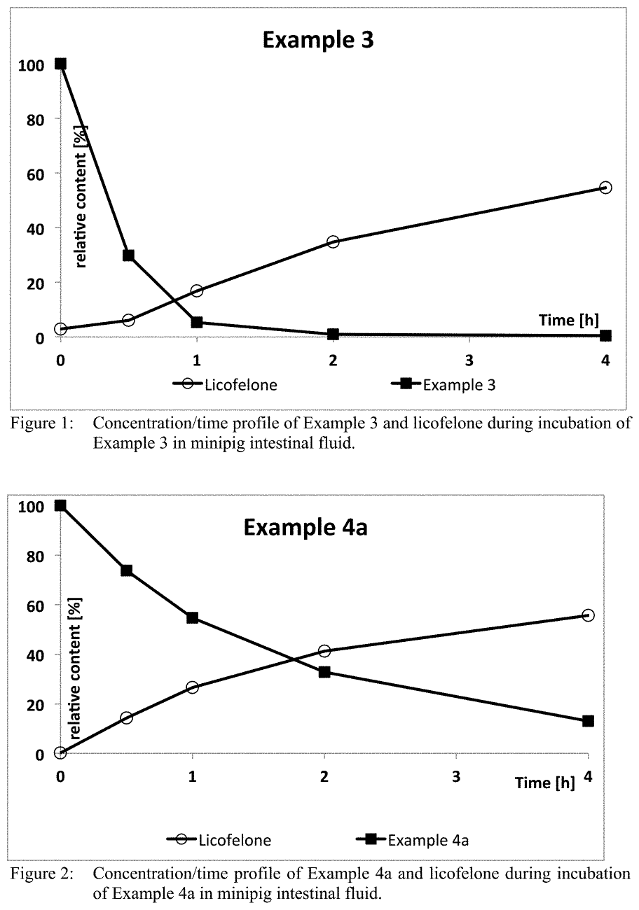

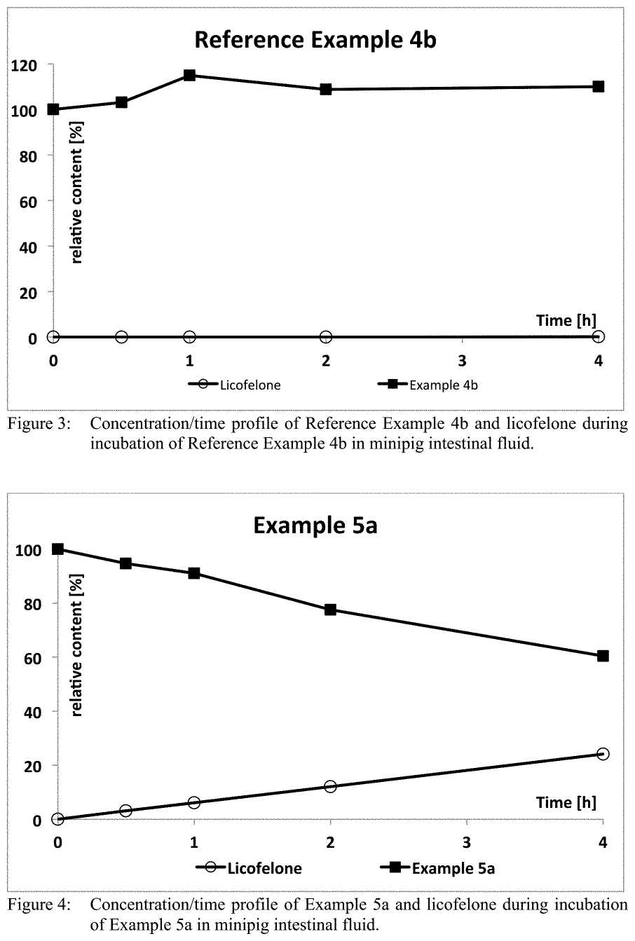

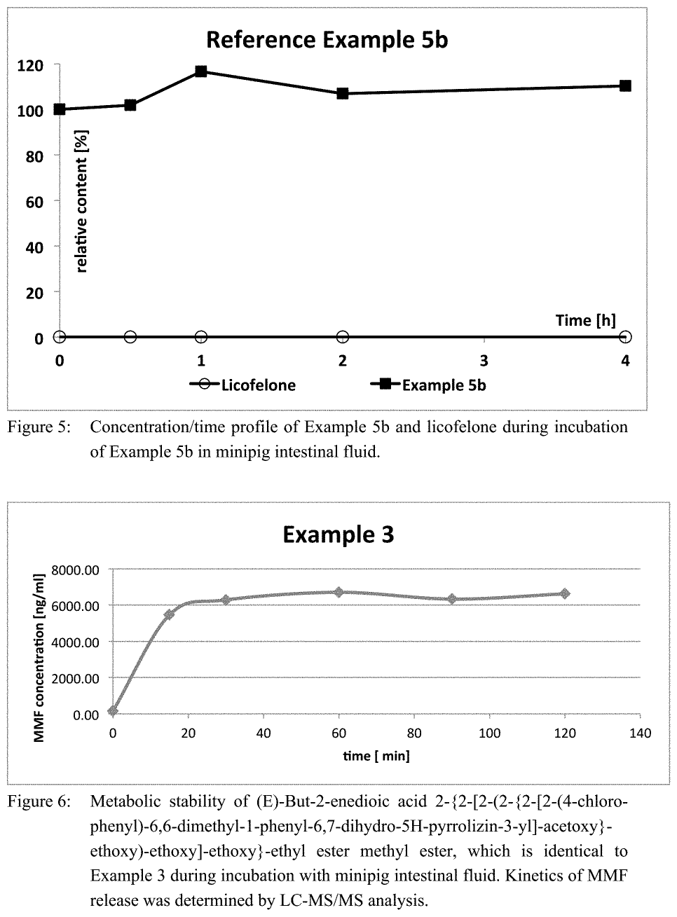

The analytical method allowed for quantification of the release of licofelone and the results for all tested compounds are summarized in the table below. During the incubation of the compound, the formation of the “licofelone-linker” intermediate was observed. Concentrations of these intermediates as well as the starting materials were semi-quantified based on the calibration established for licofelone. Concentration vs. time profiles are presented in individual figures.

| | Time [h] | | | | |
|---|---|---|---|---|---|
| | 0.5 h | 1 h | 2h | 4 h | Conc./time profile |
| Example 3 | 6.1 | 16.9 | 34.8 | 54.7 | Figure 1 |
| Example 4a | 14.4 | 26.5 | 41.2 | 55.7 | Figure 2 |
| Reference | 0.0 | 0.0 | 0.1 | 0.3 | Figure 3 |
| Example 4b | | | | | |
| Example 5a | 3.1 | 6.1 | 12.1 | 24.2 | Figure 4 |
| Reference | 0.0 | 0.0 | 0.0 | 0.1 | Figure 5 |
| Example 5b | | | | | |

As it can be seen, Reference Examples 4b and 5b, though bearing two licofelone residues, show a release of licofelone being significantly lower than the Examples bearing as residue R either a hydroxy group or an MMF residue.

### Example 10

### Metabolic Stability of Prodrugs in Minipig Intestinal Fluid—Quantification of Monomethyl Fumarate

a) Incubation Experiments with Minipig Intestinal Fluid

In a HPLC glass vial, 8 μl of stock solution were mixed with 792 μl dil IF and the mixture was stirred (250 rpm) in a water bath (T=37° C.).

Immediately after mixing as well as at t=15 min, 30 min, 60 min, 90 min and 120 min, 50 μl were withdrawn and prepared for LC-MS analysis.

Incubations were continued and in case the result of the analysis of the 120 min measurement indicated the presence of remaining intact MMF prodrug, additional samples were taken (t=360 or 420 min and at 1,260 or 1,320 min) and analyzed.

### b) Quantification of MMF by LC-MS

MMF in intestinal fluid was quantified by means of a validated LC-MS/MS method. Prior to the analysis of test samples, a calibration curve was established. Each calibration solution was analyzed two-fold. The second analysis was carried out approx. 18 h after storage of the sample in the autosampler, which was cooled to 8° C. The results demonstrate that the ratio of peak areas remains essentially unchanged between the first and the second analysis. The concentration/peak area ratio data pairs were subjected to regression analysis with 1/× weighting and the resulting calibration equation was used to quantify the MMF content in incubation samples. As Internal Standard (ISTD), monomethyl fumarate was used.

Analyses were developed and performed on an Acquity UPLC system coupled with a TQ detector (triple quadruple mass spectrometer).
Column: Phenomenex Kinetex C18, 100A, 2.6 μm (150×4.6 mm)
Flow: 0.4 ml/min
Split: appr. 100 μl/min to MS
Temperature: 30° C.
Solvent system (isocratic):
   Solvent A 25% water with 0.1% acetic acid
   Solvent B 75% methanol with 0.1% acetic acid
Stoptime: 6 min
Autosampler temperature: 8° C.
Injection volume: 4
Retention time:
   MMF: 4.3 min
   DMF: 4.7min

### Mass Spectrometry

software: Masslynx 4.1
detection mode: electrospray/negative ions (ESP−)
capillary voltage: 2.3 kV
source temperature: 100° C.
desolvation temperature: 450° C.
cone voltage: 18 V
desolvation gas: N₂, 650 L/h
cone gas: N₂, 20 L/h
collision gas: argon, appr. 3.3*10⁻³ mbar
collision energy: 11 eV
MRM [m/z]: 128.94>85.03 Monomethyl fumarate dwell: 200 msec
   142.99>99.06 Monoethyl fumarate (ISTD)
   dwell:200 msec

### Sample Preparation

50 μl sample (calibration solution or sample of an incubation experiment with MMF prodrugs) was mixed with 50 μl WS_{ISTD}, 20 μl formic acid and 100 μl acetonitrile. This mixture was vortexed for 15 sec and centrifuged (13,000 rpm, 3 min). Thereafter, 4 μl of the supernatant were subjected to LC-MS analysis.

The release of MMF is shown in FIG. 6.

### Example 11

### Investigation of the Effect of Test Compound in Experimental Encephalomyelitis in the Mouse

Assessment of the efficacy of compounds of the invention in MOG₃₅₋₅₅-induced experimental autoimmune encephalomyelitis (EAE) in C57BL/6 mice:
Test system: male C57BL/6 mice, 12 weeks old; 10 animals per treatment group;
Induction of EAE: Day −1—subcutaneous injection of MOG35-55, suspended in complete Freund's adjuvans and intraperitoneal injection of pertussis toxin.
   Day 3—intraperitoneal injection of pertussis toxin.
Treatment: Compound according to Example 3 and test substances or vehicle only were administered via oral route. Test substances were dissolved or suspended in 0.5% hydroxyethylcellulose (dissolved in 50 mM potassium dihydrogenphosphate, pH 5.0). Drug concentration in dose formulations: 11.54 mM;
Dose volume: 10 ml/kg body weight;
Start of treatment: Day 1
Observations (clinical score and body weight): Observations were recorded daily between day 1 and 13.
Clinical Score: grade 0-10; 0 (=no impairments), 1 (normal movement; limp tail: proximal 2/3 of the tail is limp and droopy), 2 (normal movement; whole tail is limp; 3 (wobbly walk; absent righting reflex), 4 (gait ataxia), 5 (mild paraparesis), 6 (moderate paraparesis), 7 (severe paraparesis or paraplegia), 8 (tetraparesis), 9 (moribund), 10 (death).

### Results:

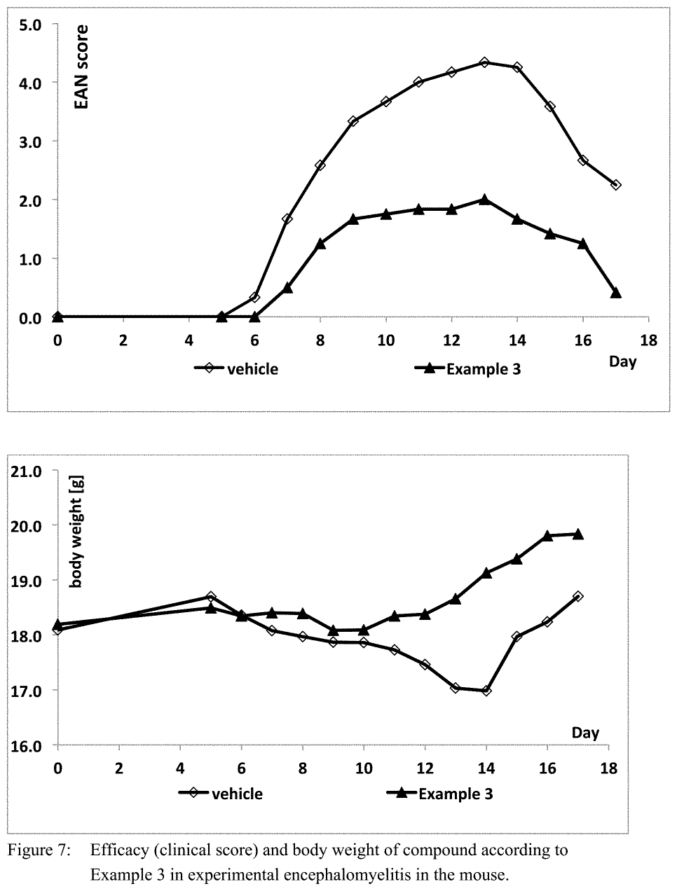

The result of the treatment with the compound according to Example 3 is shown in FIG. 7. The treatment was effective.

1. Compound according to Formula (I) 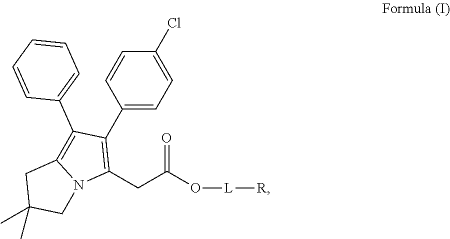
wherein
L is a linear organic residue comprising 2 to 30 carbon atoms, and
R is —OH or —OOCR′, wherein —OOCR′ is a carboxylate group which is hydrolizable during small intestinal transit.

2. Compound according to claim 1, wherein L can be a substituted or unsubstituted divalent aliphatic residue with 2 to 30 carbon atoms.

3. Compound according to claim 1, wherein L can be a substituted or unsubstituted alkylene group with 3 to 30 carbon atoms or a substituted or unsubstituted alkenylene group with 3 to 30 carbon atoms, wherein in the substituted or unsubstituted alkylene group with 3 to 30 carbon atoms or in the substituted or unsubstituted alkylene group with 3 to 30 carbon atoms one or more —CH₂- group(s) can be substituted by an oxygen atom (—O-) to form an ether.

4. Compound according to claim 1, wherein L is —((CH₂)₂O)ₘ-(CH₂)₂- wherein m is 0 to 10.

5. Compound according to claim 4, wherein m is 3.

6. Compound according to Formula (I), wherein L is defined according to claim 1 and wherein —OOCR′ is the carboxylate of monocarboxylic acids, preferably monocarboxylic acids having 2 to 6 carbon atoms, or the carboxylate of dicarboxylic acids, preferably dicarboxylic acids having 3 to 10 carbon atoms, and derivatives, preferably monoesters, thereof.

7. Compound according to claim 1, wherein —OOCR′ is the carboxylate of monomethyl fumaric acid.

8. Compound according to claim 1 according to Formula (V)

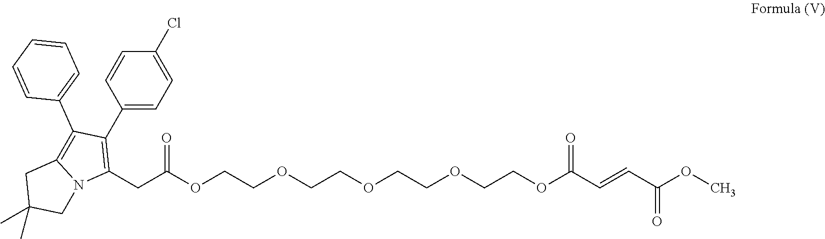

9. Compound according to claim 1 for use as a medicament.

10. Compound according to claim 1 for use in the treatment of systemic diseases, autoimmune diseases or inflammatory diseases, preferably for the use in the treatment of osteoarthritis and/or multiple sclerosis, rheumatoid arthritis or psoriasis.

11. Pharmaceutical composition comprising a compound according to claim 1.

12. Pharmaceutical composition comprising
(i) 0.01 to 10 mmol of a compound according to claim 1 and
(ii) optionally pharmaceutical excipients.

13. Pharmaceutical composition according to claim 11, wherein the composition is a solid oral dosage form.

14. Method for treating and/or preventing systemic diseases, autoimmune diseases and/or inflammatory diseases, preferably osteoarthritis and/or multiple sclerosis, rheumatoid arthritis or psoriasis, in particular multiple sclerosis, comprising administering to a subject in need thereof a therapeutically effective amount of the compound according to claim 1.

15. Method for treating and/or preventing systemic diseases, autoimmune diseases and/or inflammatory diseases, preferably osteoarthritis and/or multiple sclerosis, rheumatoid arthritis or psoriasis, in particular multiple sclerosis, comprising administering to a subject in need thereof a therapeutically effective amount of the pharmaceutical composition according to claim 11.
